(19) United States
(12) Patent Application Publication
(10) Pub. No.: US 2011/0275114 A1
(43) Pub. Date: Nov. 10, 2011

(54) METHOD AND MEDIUM FOR DETECTING THE PRESENCE OR ABSENCE OF METHICILLIN RESISTANT STAPHYLOCOCCUS AUREUS (MRSA) IN A TEST SAMPLE

(76) Inventor: Stephen C. Edberg, Belleair Bluffs, FL (US)

(21) Appl. No.: 12/775,236

(22) Filed: May 6, 2010

(51) Int. Cl. C12Q 1/14

(52) U.S. Cl. 435/36

(57) ABSTRACT

A dry sample analyzing mixture, a liquid sample analyzing medium, and a sample analyzing method, are described for use in detecting the presence or absence of Methicillin Resistant Staphylococcus Aureus (“MRSA”) in an incubated first generation biological or environmental specimen sample. First generation specimen samples that can be analyzed include nasal swabs, lesion swabs, skin swabs, throat swabs, food swabs, tanning salon swabs, gym swabs, restaurant swabs, and the like. The medium and method include an anti-ribosomal antibiotic component that will selectively prevent Methicillin Susceptible Staphylococcus Aureus (“MSSA”) from growing in the medium, while allowing MRSA to grow in the medium. The medium also includes components which will stimulate growth of MRSA. The medium also includes components which will produce a detectable signal, which signal indicates the presence of MRSA in the incubated sample.

TECHNICAL FIELD

The present method and testing medium relate to the detection of Staphylococcus aureus in a biological, environmental, or food sample, and more particularly to those methods and testing media utilizing reacting factors with which the target microbe(s) can produce a detectable signal in a hydrated mixture of the medium and sample being tested. Ingredients which can prevent false positive results for the presence of Methicillin Resistant Staphylococcus aureus (MRSA) in the sample are included in the testing medium. Examples of suitable ingredients include, but are not limited to, amino glycoside anti-ribosomal antibiotics, such as gentamicin, tobramycin, and kanamycin, for example, which are active against MSSA but not MRSA. When used in conjunction with a cell wall active anti microbial agent, the anti-ribosomal antibiotic enhances the detection of MRSA.

### BACKGROUND INFORMATION

Staphylococcus aureus (S. aureus) can be a virulent pathogen of animals and humans. Moreover, it can cause severe food poisoning by the production of a toxin. Diseases caused by S. aureus cover a very wide clinical spectrum, from simple skin infections to life threatening infections of the bones, heart, and organs. Of particular concern is the recognition that S. aureus infection is common after surgery. It is also associated with intravenous tubing and other implants.

The bacterium S. aureus may be transmitted between healthy individuals by skin to skin contact, or from a commonly shared item or a surface (e g , tanning beds, gym equipment, food handling equipment, etc.) where the transfer may be made to a subsequent person who uses the shared item or touches the surface. Of great medical concern is the recognition that healthy people entering hospitals may “carry” S. aureus (e.g., on their skin, or in their noses, etc.) without any signs or symptoms that they do so. In the presence of favorable conditions (often found in but not limited to hospitals), the S. aureus can activate and cause serious infection. In addition, S. aureus can also be a source of food poisoning, often caused by a food handler contaminating the food product (e.g., meat, poultry, eggs, salads containing mayonnaise, bakery products, dairy products, etc.).

There are two categories of S. aureus based on an individual clone's susceptibility to the class of antibiotics that began with methicillin. These are methicillin susceptible S. aureus (MSSA), and methicillin resistant S. aureus (MRSA). Until only a few years ago, MRSA was most commonly found in hospitals. Now, it is frequently also present in the noses, skin, etc. of people in the non-hospital community. Moreover, these MRSA bacteria are increasingly causing serious infections in the community. MRSA is particularly serious because only very few antibiotics (e.g., vancomycin) have been shown to be uniformly effective against MRSA.

The Center for Disease Control and Prevention actively surveys for the development of methicillin resistant S. aureus. In 2000, the Society for Healthcare Epidemiology of America guidelines recommended contact isolation for patients with MRSA. In addition to the morbidity and mortality caused by MRSA, it has been estimated that each case of infection costs at least $23,000. Accordingly, many hospitals and nursing homes proactively sample patients for MRSA [Clany, M., Active Screening in High-Risk units is an effective and cost-avoidant method to reduce the rate of methicillin-resistant Staphylococcus aureus infection in the hospital, Infection Control and Hospital Epidemiology 27: 1009-1017, 2006].

Meyer et al. (U.S. Pat. No. 4,035,238) describes the use of a broth for the detection of S. aureus that utilizes mannitol as a source of carbon and DNA meth. Neither of these chemicals are coagulase reactive substrates.

Rambach (U.S. Pat No. 6,548,268) employs at least two chromogenic agents in an agar medium: 5-bromo-6-chloro-indoxyl-phosphate; and 5-bromo-4-chloro-3-indoxyl glucose in the presence of desferoxamine. An individual colony hydrolyzing these substrates will produce colors that will mix with each other and not be independent of one another.

A large number of classical culturing procedures are utilized to detect MSSA and MRSA from human, animal, food, etc. samples. They have in common a basic medium with chemical inhibitors such as 6-8% sodium chloride, potassium tellurite, and a variety of antibiotics. For example Stevens and Jones described the use of a trehalose-mannitolphosphatase agar [Stevens, D L and Jones, C. “Use of trehalose-mannitol-phosphatase agar to differentiate Staphylococcus epidermidis and Staphylococcus saprophyticus from other coagulase-negative staphylococci”, J. of Clin. Microbiology 20:977-980, 1984]. The use of mannitol as a carbon source and salt as a selective agent into an agar known as mannitol-salt agar has been commonly used in clinical laboratories [Baird, R. M. and W. H. Lee., Media used in the detection and enumeration of Staphylococcus aureus., Int. J. Food Microbiology. 26:209-211, 1995]. Within the prior art of culturing, it is a generally accepted procedure to perform coagulase tests utilizing samples of S. aureus that are isolated in a pure culture as a required test to achieve sufficient specificity.

The procedure “S. aureus 10” [Bio Merieux, La Balme Les Grottes, France] uses an alpha-glucosidase substrate in agar to detect S. aureus. A single substrate is utilized. [Perry, J. D. et al., “Evaluation of S. aureus 10, a new chromogenic agar medium for detection of Staphylococcus aureus”, J. Clin. Microbiology 41 :5695-5698, 2003]. A variant of this medium, which contains added antibiotics and sodium chloride, is designed to detect MRSA [Perry et al., “Development and evaluation of a chromogenic agar medium for methicillin-resistant Staphylococcus aureus”, J. of Clin. Micro. 42:4519-4523, 2004].

It would, therefore, be desirable to provide a test mixture and a method that can rapidly detect MRSA directly from a first generation sample, one that does not require a skilled technician to perform the method, one that can be performed without the need to develop isolates from the specimen sample (i.e., one that can be performed on a “first generation” specimen sample), and one that does not require a large concentration of S. aureus organisms to be accurate, and one that is stable at room temperatures for an extended time period.

### SUMMARY OF THE INVENTION

This invention relates to a method and test mixture for specific detection of MRSA bacteria in a first generation biological, environmental, or food sample. In the detection of MRSA, a test mixture (or “medium”) is utilized. The medium will include growth inhibitors which will inhibit growth of MSSA but will not inhibit growth of MRSA. The medium will also include a pH control and a nutrient indicator, or a specifically metabolizable substrate that will promote growth of MRSA and will produce a detectable signal in the test sample/medium mixture if MRSA is present in the test sample. If MRSA is not present in the test sample, no detectable signal will be produced. The detectable signal will typically be produced within 6 to 8 hours after inoculation of the medium with the sample.

As noted above, MSSA growth inhibitors are included in the medium to inhibit or otherwise negatively affect MSSA bacterial growth, while not interfering with MRSA bacterial growth. The untreated sample is added to the test mixture, and the resultant inoculated test sample is incubated. The MSSA growth inhibitors can include Cefoxitin, Colistin, Aztreonam, Gentamicin, Kanamycin or Sisomycin, for example.

The test mixture is preferably prepared in a form that facilitates handling, packaging, storing, etc., of the test mixture. A dry powder that can be hydrated into liquid form is a particularly preferable embodiment of the test mixture, but the present invention is not limited to a powder faun. The test mixture may assume a liquid form, or any other form (e.g., paste, gel, etc.), preferably one that can be hydrated for use.

The growth promoting constituents within the test mixture that facilitate the multiplication of and sustain S. aureus can be varied to suit the application. Those in the art will recognize that many different combinations of constituents, and varying relative amounts of the same constituents, can be used to provide the same functionality. Growth promoting constituents include sources of nitrates and proteins; materials operative to assist in the generation of nucleic acid synthesis; sources of energy for the S. aureus; sources of amino acid growth factors; and, in some embodiments, materials operable to help repair damaged target organisms. This list of growth promoting constituents does not represent all of the materials that can be beneficial within the test mixture, but does illustrate materials that are acceptable (e.g., vitamins, salts, minerals, inorganic moieties, etc.). The test mixture may include other constituents that benefit the performance of the test mixture.

Of particular utility is the use of anti-ribosomal amino glycoside antibiotics, such as, gentamicin, kanamycin, tobramycin, and sisomicin to inhibit MSSA, but not MRSA.

In most applications of the present invention, it will be desirable to utilize a test mixture that includes the following: a) an effective amount of amino acids; b) an effective amount of nitrogen sources; c) an effective amount of salts; d) an effective amount of vitamins; e) an effective amount of calcium; and an effective amount of a hydrolyzable substrate, such as one or more sugars that can be metabolized by MRSA. Those skilled in the art will recognize that natural sources of such amino acids can be used rather than pure sources. The natural sources (e.g. extract of whole organisms, such as yeast) may be in mixture form or in purified form. The natural mixtures can contain varying amounts of such amino acids and vitamins Those skilled in the art will further recognize that many different combinations of amino acids and vitamins can be used in the present invention test mixture. Effective amounts of drug ingredients which selectively inhibit the growth of MSSA in the sample are also included in the testing mixture. As noted above, examples of such suitable drug ingredients include the anti-ribosomal antibiotic gentamicin, which is active against MSSA but not MRSA. Drugs in this class can stop protein synthesis.

Those in the art will further recognize that carbon, nitrogen, trace elements, vitamins, amino acids and selective agents can be provided in many forms. Generally, it is preferred to have an amount of vitamins and amino acids within a predetermined range, but those in the art will recognize that the actual properties of each ingredient may be varied so that reduction in the amount of one ingredient can be compensated by an increase in the amount of another. This is particularly relevant when the essential amino acids, trace elements or vitamins of the microbes sought to be detected are known. Some ingredients may be provided in reduced amounts or deleted if they may be synthesized endogenously by the microorganism whose presence is to be determined Salts may be provided as a source of ions upon dissociation.

The test mixture may be packaged in a container (e.g., a test tube, a container with a flat bottom wall, etc.) that facilitates the testing process. If the medium is prepared in a form that can be hydrated, the mixture can be hydrated with sterile water or non-sterile water.

To detect the presence of MRSA within a sample, the sample is obtained from a biological, environmental, or food specimen. A sample collected using a nasal swab is an example of a first generation sample that is particularly convenient to collect and test using the present invention. Once collected, the sample is inoculated into the test mixture.

The inoculated sample is incubated under conditions favorable to facilitate the multiplication of MRSA that may be present within the inoculated sample, while suppressing the multiplication of MSSA that may be present in the sample. In the case of a powdered test mixture hydrated with water, the incubation may be carried out at temperatures between about 20° C. to 35° C. The combination of sequential enzyme specificity, MRSA enhancing growth factors, and an MSSA suppressing antibiotic, selectivity, provides multiple hurdles which prevent the competing non-target bacteria from being detected within the test period; e.g., a period of 24 hours or less.

The present invention testing paraphernalia and method can be used in hospital admissions, routinely in intensive care units, in nursing homes, dialysis patients, people receiving home immunosuppressive therapy, and the like. It can also be used in environmental settings (e.g., gyms, tanning salons, restaurants, etc.) where the bacteria MRSA may be transferred from a human carrier and it can be used to test various different foods for MRSA contamination. It will be appreciated that a substantial benefit of the present method and mixture is that they may be performed/used without the need for expensive equipment or skilled medical technologists, Another substantial benefit of the present method/mixture is that it is operative to detect a relatively small amount of MRSA in the test sample; e.g., the present method/mixture has detected MRSA in samples having concentrations of MRSA as low as 100 CFU/ml.

### BRIEF DESCRIPTION OF THE DRAWINGS

Details of the invention will become more readily apparent from the following detailed description of several embodiments of the invention when taken in conjunction with the accompanying drawings in which:

FIG. 1 is a side elevational view of a test tube containing a dry test mixture which is to be used in performing the MRSA presence or absence testing procedure of this invention;

FIG. 2 is a side elevational view of a set of three test tubes of the type shown in FIG. 1 after the test mixture in each tube has been hydrated;

FIG. 3 is a side elevational view of the set of test tubes of FIG. 2 after the test has been performed on a sample specimen, wherein the sample specimen has been found to be free of MRSA; and

FIG. 4 is a side elevational view of the set of test tubes of FIG. 2 after the test has been performed on a sample specimen, wherein the sample specimen has been found to contain MRSA.

### DETAILED DESCRIPTION OF SPECIFIC EMBODIMENTS OF THE INVENTION

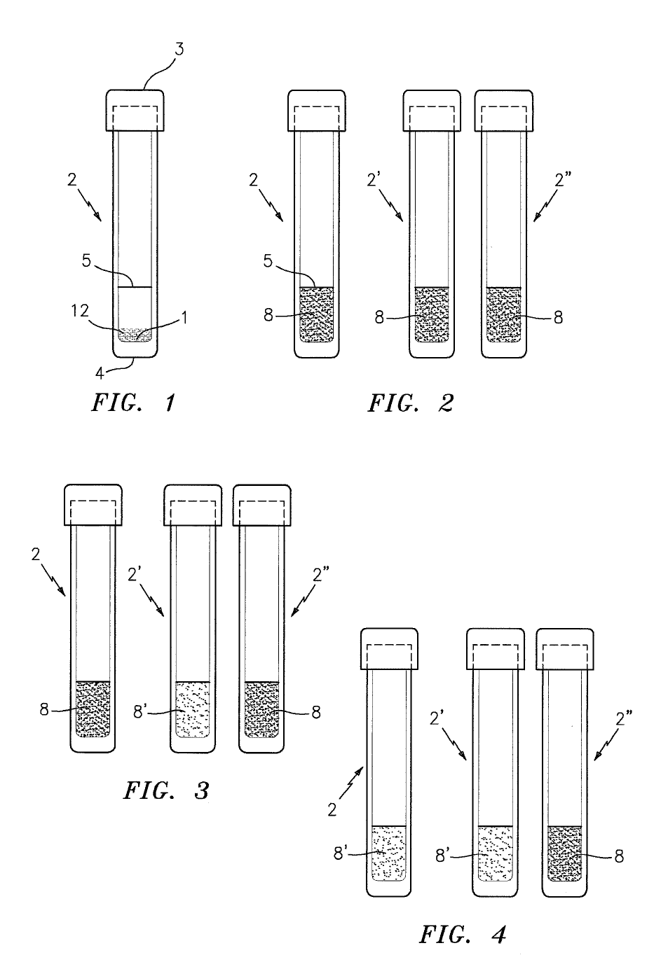

FIG. 1 is a side elevational view of a test tube denoted by the numeral 2 which contains a sample test mixture 12 for use in performing the MRSA presence/absence test of this invention. The tube 2 preferably has a flat bottom 4 and a top closure 3. The tube 2 contains a dry powdered test mixture 12 which is formed in accordance with this invention for detecting the presence or absence of MRSA in a sample; e.g., a first generational biological sample. The tube 2 is also provided with a reference line 5 which indicates the amount of hydrating liquid, preferably water, to be added to the tube 2 in order to properly hydrate the powdered mixture 12 for specimen sample testing.

An effective formulation for detecting the presence or absence of MRSA in a first generation sample of the type referred to herein is set forth below. The amounts of each ingredient in the formulation are found to be effective amounts thereof

| | Gms/L of | | |
|---|---|---|---|
| Constituent | Test Mixture | Range | Function |
| MuellerHinton Broth | 8.00 | 1.0-20.0 | Protein source |
| Yeast Extract | 5.25 | 0.5-20.0 | Vitamin source |
| Lithium Chloride | 5.0 | 1.0-10.0 | Selective agent |
| Trehalose | 12.0 | 1.0-20.0 | Source of carbon |
| Phenol red | 0.018 | 0.01-0.03 | pH indicator |
| Maltose | 3.0 | 0.05-10.0 | inducer sugar |
| Mannitol | 5.0 | 0.055-20.0 | second carbon |
| | | | source |
| Amphotericin B | 0.005 | 0.001-0.020 | Anti-yeast sel. |
| | | | agent |
| Kinetin | 0.001 | 0.001-0.010 | P.G. Hormone |
| Indol acetic acid | 0.005 | 0.001-0.020 | P.G. Hormone |
| Indol butyric acid | 0.005 | 0.001-0.020 | ″ |
| Gibberellic acid | 0.001 | 0.0001-0.020 | P.G. Hormone |
| IPTG | 0.005 | 0.0001-0.020 | inducer |
| Phenyl ethyl alcohol | 1.500 ml | 0.5 ml-10 ml | anti-gram |
| | | | negative |
| Desfuroxime | 0.0015 | 0.0005-0.0050 | |
| Potassium phosphate | 0.500 | 0.10-5. | effervescent |
| Aztreonam | 0.020 | 0.005-0.050 | antibiotic |
| Colistin | 0.005 | 0.0011-0.020 | ″ |
| Cefoxitin | 0.008 | 0.001-0.030 | selective |
| | | | antibiotic |
| Kanamycin | 0.030 | 0.001-0.050 | selective |
| | | | antibiotic |

Gentamicin and/or Tobramycin in an amount of 0.010 and in a range of 0.001-0.050 Gms/L can be substituted for Kanamycin in the above test mixture formulation. This specific example does not represent all test mixture formulations, and the present invention is not limited thereto. As stated above, those in the art will recognize that many different combinations of constituents, and varying effective amounts of the same, can be used to provide the same functionality. Hence, the present method and mixture contemplates that a number of different constituent formulations can be made. The above mixture preferably includes an effective amount of a protein source; an effective amount of a vitamin source; an effective amount of a carbon source; an effective amount of plant hormones; an effective amount of a pH indicator; and a selective amount of an antibiotic which is directed against MSSA.

FIG. 2 shows three of the test tubes 2,2′ and 2″ wherein the powdered mixture 12 has been properly hydrated by the addition of water, preferably distilled water, to form a hydrated test mixture 8. The tube 2 is the sampling tube to which a first generation specimen sample to be analyzed for the presence or absence of MRSA is added. The specimen sample, which can be a swab of the subject being tested, is combined with the hydrated test mixture 8. The tubes 2′ and 2″ are tubes of the hydrated test mixture which are used as positive and negative controls for the test mixture. When performing the sample analyzation procedure, the sample being tested is added to the hydrated test mixture 8 in the tube 2, while a sample of MRSA is added to tube 2′ and a sample of MSSA is added to tube 2″. In the instant case, when the test mixture is hydrated, the hydrated solution has a particular color which can be red for example. In the example shown in the drawings the initial color is a dark color, such as red. FIG. 3 shows one result of the test after a predetermined incubation period which can be from eight to twenty four hours, for example. In the tube 2 in which the sample being analyzed was placed, there is no color change in the hydrated mixture 8, while in the positive control tube 2′ to which the MRSA was added, the color of the hydrated mixture 8′ has changed and become lighter. This is a confirmation of the presence of MRSA in the mixture 8′. It will be noted also that the hydrated test mixture 8 in the tube 2″ to which the MSSA was added did not Change color. Thus the specimen tube 2 indicates no presence of MRSA while the tube 2′ does and the tube 2″ does not. Thus this test result indicates that the specimen sample being tested is free of MRSA. FIG. 4 indicates that the specimen being tested in the tube 2 does contain MRSA since the hydrated specimen sample test mixture changes to the color 8′ which is the same as the color of the test mixture 8′ in the positive control tube 2′. This color change indicates that the specimen sample does contain MRSA.

First generational test samples can be collected by a variety of different techniques; e.g., a human sample can be collected by wiping a swab within the nose of a subject. Nasal swabs are a particularly convenient way of collecting a test sample, but they are not the only collection method; e.g., test samples can be collected from throat swabs, skin lesions, undamaged skin, etc. First generational environmental samples can be collected by various known methods for example, by wiping or swabbing a surface using a dry or wet wipe/swab. Likewise, first generational food samples can be collected from the food itself, or wiping food residue from surfaces in contact with the food, etc. Once the sample is collected, it can be deposited in the hydrated test mixture 6 by using the same cotton swab 8 which has been used to gather the first generation sample from the source thereof. Once the specimen sample is deposited in the test mixture 6, it is incubated within the test mixture for a period of time typically less than twenty-four hours. The incubation may occur at any temperature that is acceptable under the circumstances.

In addition to the above formulation, a control formulation which will rule out false positive results can be included in test kits for performing the analysis. The control formulation will be the same as that set forth above with the exception that it will not include Cefoxitin.

While the invention has been described with respect to preferred embodiments, those skilled in the art will readily appreciate that various changes and/or modifications can be made to the invention.

What is claimed is:

1. A method for detecting the presence or absence of Methicillin Resistant Staphylococcus aureus (“MRSA”) in a first generation biological, food, or environmental specimen sample, said method comprising the steps of:
a) providing a powdered reagent, said reagent containing at least one antibiotic component which will selectively inhibit the growth of Methicillin Susceptible Staphylococcus aureus (“MSSA”); one or more hydrolyzable substrates which MRSA can metabolize and MSSA cannot; and a substance that can be metabolized by MRSA to produce a detectable signal in a test tube containing said specimen sample;
b) hydrating said reagent in said test tube;
c) forming a mixture of said first generation specimen sample and said hydrated reagent in said test tube;
d) incubating said mixture of said hydrated reagent and said specimen sample in said tube at temperatures in the range of about 20° C. to about 35° C.; and
e) observing said mixture to note the presence or absence of said detectable signal in said mixture in said tube.

2. The method of claim 1 wherein said reagent includes S. aureus growth promoting factors including:
a) an effective amount of amino acids;
b) an effective amount of nitrogen sources;
c) an effective amount of salts;
d) an effective amount of vitamins;
e) an effective amount of calcium;
f) an effective amount of protein;
g) an effective amount of a hydrolyzable substrate which only MRSA can metabolize; and
h) an effective amount of sugars which MRSA can metabolize.

3. The method of claim 1 wherein said reagent includes S. aureus growth promoting factors including:
a) an effective amount of amino acids;
b) an effective amount of nitrogen sources;
c) an effective amount of salts;
d) an effective amount of vitamins;
e) an effective amount of calcium;
f) an effective amount of protein;
g) an effective amount of a hydrolyzable substrate which only MRSA can metabolize;
and h) an effective amount of a hydrolyzable substrate which MRSA can metabolize.

4. The method of claim 1 wherein said reagent includes S. aureus growth promoting factors including:
a) an effective amount of amino acids;
b) an effective amount of nitrogen sources;
c) an effective amount of salts;
d) an effective amount of vitamins;
e) an effective amount of calcium;
f) an effective amount of protein;
g) an effective amount of a hydrolyzable substrate which only MRSA can metabolize;
and h) an effective amount of amino acids which MRSA can metabolize.

5. The method of claim 1 wherein said antibiotic component includes an effective amount of an anti-ribosomal antibiotic or anti-metabolite which will selectively inhibit any MSSA in the specimen sample.

6. The method of claim 5 wherein said anti-ribosomal antibiotic is selected from the group consisting of: gentamicin, tobramycin, kanamycin, and mixtures thereof

7. The method of claim 5 wherein said anti-ribosomal antibiotic is kanamycin.

8. The method of claim 5 wherein said anti-ribosomal antibiotic is tobramycin.

9. The method of claim 1 wherein said specimen sample is a biological sample such as a nasal swab, a lesion swab, a skin swab, a throat swab, or the like.

10. The method of claim 1 wherein said specimen sample is an environmental sample such as a tanning salon swab, a gym swab, a restaurant swab, or the like.

11. A liquid medium for detecting the presence or absence of Methicillin Resistant Staphylococcus aureus (“MRSA”) in a first generation biological, food, or environmental specimen sample, said medium comprising:
a) an effective amount of amino acids;
b) an effective amount of nitrogen sources;
c) an effective amount of salts;
d) an effective amount of vitamins;
e) an effective amount of calcium;
f) an effective amount of protein; and
g) an effective amount of at least one antibiotic component which will selectively inhibit the growth of Methicillin Susceptible Staphylococcus aureus (“MSSA”).

12. The medium of claim 11 wherein said antibiotic component includes an effective amount of an anti-ribsomal antibiotic which will selectively inhibit any MSSA in the specimen sample.

13. The medium of claim 12 wherein said anti-ribosomal antibiotic is selected from the group consisting of: gentamicin, tobramycin, kanamycin, and mixtures thereof.

14. The medium of claim 12 wherein said anti-ribosomal antibiotic is tobramycin.

15. The medium of claim 11 wherein said specimen sample is a biological sample selected from the group consisting of: a nasal swab, a lesion swab, a skin swab, a throat swab, saliva; and
the like.

16. The medium of claim 11 wherein said specimen sample is an environmental sample selected from the group consisting of: a tanning salon swab, a gym swab, a restaurant swab, or the like.

17. A dry mixture of ingredients for use in detecting the presence or absence of Methicillin Resistant Staphylococcus aureus (“MRSA”) in a first generation biological, food, or environmental specimen sample, said mixture of ingredients comprising:
a) an effective amount of amino acids;
b) an effective amount of nitrogen sources;
c) an effective amount of salts;
d) an effective amount of vitamins;
e) an effective amount of calcium;
f) an effective amount of protein; and
g) an effective amount of at least one antibiotic component which will selectively inhibit the growth of Methicillin Susceptible Staphylococcus Aureus (“MSSA”).

18. The mixture of ingredients of claim 17 wherein said antibiotic component includes an effective amount of an anti-ribsomal antibiotic which will selectively inhibit any MSSA in the specimen sample.

19. The mixture of ingredients of claim 17 wherein said anti-ribosomal antibiotic is selected from the group consisting of: gentamicin, tobramycin, kanamycin, and mixtures thereof.

20. The mixture of ingredients of claim 17 wherein said anti-ribosomal antibiotic is tobramycin.

21. A method for detecting the presence or absence of Methicillin Resistant Staphylococcus aureus (“MRSA”) in a first generation biological, food, or environmental specimen sample, said method comprising the steps of:
a) providing a reagent which contains at least one antibiotic component which will selectively inhibit the growth of Methicillin Susceptible Staphylococcus aureus (“MSSA”) and substrates that can be metabolized by MRSA to produce a detectable signal in a test tube containing said specimen sample;
b) said reagent also containing:
i) an effective amount of amino acids;
ii) an effective amount of nitrogen sources;
iii) an effective amount of salts;
iv) an effective amount of vitamins;
v) an effective amount of calcium;
vi) an effective amount of protein; and
vii) an effective amount of a substrate or substrates which MRSA can selectively metabolize;
c) hydrating said reagent in said test tube;
d) forming a mixture of said first generation specimen sample and said hydrated reagent in said test tube;
e) incubating said mixture in said tube at temperatures in the range of about 20° C. to about 35° C.; and
f) observing said mixture to note the presence or absence of said detectable signal in said mixture in said tube.
